(19) United States
(12) Patent Application Publication
(10) Pub. No.: US 2026/0091004 A1
(43) Pub. Date: Apr. 2, 2026

(54) PHARMACEUTICAL INJECTABLE SOLUTION COMPRISING DOPAMINE

(71) Applicants: INBRAIN PHARMA, LOOS (FR); UNIVERSITE DE LILLE, LILLE (FR); CENTRE HOSPITALIER UNIVERSITAIRE DE LILLE, LILLE (FR); INSTITUT NATIONAL DE LA SANTE ET DE LA RECHERCHE MEDICALE, Paris (FR)

(72) Inventors: Pascal ODOU, GODEWAERSVELDE (FR); David DEVOS, MARCQ-EN-BAROEUL (FR); Alexandre DEMAILLY, BIENVILLERS-AU-BOIS (FR); Matthieu FISICHELLA, SANNOIS (FR)

(21) Appl. No.: 19/111,868

(22) PCT Filed: Sep. 13, 2023
(86) PCT No.: PCT/EP2023/075180

§ 371 Date: Mar. 14, 2025

(30) Foreign Application Priority Data
Sep. 14, 2022 (EP) 22306352.0

(51) Int. Cl. A61K 31/137; A61K 9/00; A61K 9/08; A61P 25/16

(52) U.S. Cl. CPC A61K 31/137; A61K 9/0085; A61K 9/08; A61P 25/16

(57) ABSTRACT

The present disclosure relates to a pharmaceutical injectable solution comprising dopamine or a pharmaceutically acceptable salt thereof, preferably dopamine hydrochloride, dissolved in water for injection, wherein the solution has a pH between 3.0 and 5.5, and has an oxygen content equal to or lower than 0.008% (8 ppm), and uses thereof.

### FIELD OF THE INVENTION

The present disclosure relates to a pharmaceutical injectable solution comprising dopamine hydrochloride dissolved in water for injection, wherein the solution has a pH between 3.0 and 5.5, and has an oxygen content equal to or lower than 0.008% (8 ppm).

### BACKGROUND OF THE INVENTION

Parkinson's disease (PD) is a progressive neurodegenerative disease affecting the nervous system, in particular the nigro-striatal system comprising dopaminergic neurons. The loss of dopamine in the striatum, as a result of progressive neuronal degeneration in the substantia nigra pars compacta (SNpc), is responsible of motor symptoms.

The pharmacologic treatment of Parkinson's disease can be divided into neuroprotective and symptomatic therapy. Neuroprotective therapy of Parkinson's disease is based on the protection of the dopaminergic neurons in the human substantia nigra and the striatum from the complex degenerative process that causes premature cell death and depletion of dopamine. In practice, however, nearly all of the available treatments are symptomatic in nature and do not appear to slow or reverse the natural course of the disease. Indeed, there is no neuroprotective treatment available on the market at the moment.

Numerous symptomatic treatments have thus focused on the attenuation of this dopamine deficiency (Chauduri et al., 2009; Devos et al. 2013) (Chaudhuri KR1, Schapira A H. Non-motor symptoms of Parkinson's disease: dopaminergic pathophysiology and treatment. Lancet Neurol. 2009; 8:464-74; Devos D, Lejeune S, Cormier-Dequaire F, Tahiri K, Charbonnier-Beaupel F, Rouaix N, Duhamel A, Sablonniére B, Bonnet A M, Bonnet C, Zahr N, Costentin J, Vidailhet M, Corvol J C. Dopa-decarboxylase gene polymorphisms affect the motor response to L-dopa in Parkinson's disease. Parkinsonism Relat Disord. 2014; 20:170-5).

As dopamine does not cross the digestive mucosa or the blood brain barrier, its lipophilic precursor L-dopa (Levodopa) has been developed as an orally administered medication in order to alleviate symptoms of Parkinson's disease.

However numerous pharmacokinetic drawbacks are related to the use of L-dopa, and trigger appearance of L-dopa related complications (LDRC). L-dopa has a short half-life in plasma and results in pulsatile dopaminergic stimulation. Under normal conditions, the dopaminergic neurons in the substantia nigra pars compacta (SNpc) fire continuously and the dopamine concentration in the striatum is maintained at a relatively constant level (Miler and Abercrombie, 1999; Venton et al., 2003; Olanow et al., 2006) (Miller D W, Abercrombie E D.; Role of high-affinity dopamine uptake and impulse activity in the appearance of extracellular dopamine in striatum after administration of exogenous L-DOPA: studies in intact and 6-hydroxydopamine-treated rats. J Neurochem. 1999; 72:1516-22; Venton B J, Zhang H, Garris P A, Phillips P E, Sulzer D, Wightman R M. Real-time decoding of dopamine concentration changes in the caudate-putamen during tonic and phasic firing. J Neurochem. 2003; 87:1284-95; Olanow C W, Obeso J A, Stocchi F. Continuous dopamine-receptor treatment of Parkinson's disease: scientific rationale and clinical implications. Lancet Neurol. 2006; 5:677-87.). In the dopamine-depleted state, however, intermittent oral doses of levodopa induce discontinuous stimulation of striatal dopamine receptors and after long-term treatment contribute to the dysfunction of the dopaminergic pathways leading to the development of motor complications (Fahn and the Parkinson study group, 2005; Parkinson study group, 2009) (Fahn S; Parkinson Study Group. Does levodopa slow or hasten the rate of progression of Parkinson's disease? J Neurol. 2005; 252 Suppl 4:IV37-IV42; Parkinson Study Group CALM Cohort Investigators. Long-term effect of initiating pramipexole vs levodopa in early Parkinson disease. Arch Neurol. 2009; 66:563-70). This oral pulsatile administration leading to alternative periods of underdosage and overdosage could contribute to the worsening of the disease progression (Devos et al., 2013). Indeed, intermittent oral administration of L-dopa is unable to restore the continuous nigro-striatal dopaminergic neurotransmission.

Continuous dopaminergic administration might be more physiologic and could prevent high fluctuations in the dopamine level inducing deleterious consequences.

Some treatments have thus focused on a continuous dopaminergic administration. However, direct delivery of a gel of levodopa to the duodenum (Olanow C W, Kieburtz K, Odin P, Espay A J, Standaert D G, Fernandez H H, Vanagunas A, Othman A A, Widnell K L, Robieson W Z, Pritchett Y, Chatamra K, Benesh J, Lenz R A, Antonini A; LCIG Horizon Study Group. Continuous intrajejunal infusion of levodopa-carbidopa intestinal gel for patients with advanced Parkinson's disease: a randomised, controlled, double-blind, double-dummy study. Lancet Neurol. 2014; 13:141-9; Devos D; French DUODOPA Study Group. Patient profile, indications, efficacy and safety of duodenal levodopa infusion in advanced Parkinson's disease. Mov Disord. 2009; 24:993-1000) or subcutaneous infusions of apomorphine, a dopamine agonist (Manson A J, Turner K, Lees A J. Apomorphine monotherapy in the treatment of refractory motor complications of Parkinson's disease: long-term follow-up study of 64 patients. Mov Disord. 2002; 17:1235-41; Drapier S, Gillioz A S, Leray E, Péron J, Rouaud T, Marchand A, Vérin M. Apomorphine infusion in advanced Parkinson's patients with subthalamic stimulation contraindications. Parkinsonism Relat Disord. 2012; 18:40-4), have shown moderate efficiency to reduce LDRC and a poor ergonomy due to external pump (Syed N, Murphy J, Zimmerman T Jr, Mark M H, Sage J I. Ten years' experience with enteral levodopa infusions for motor fluctuations in Parkinson's disease. Mov Disord. 1998; 13:336-8; Devos D; French DUODOPA Study Group. Patient profile, indications, efficacy and safety of duodenal levodopa infusion in advanced Parkinson's disease. Mov Disord. 2009; 24:993-1000). The use of long-acting dopamine agonists (Rascol O, Brooks D J, Korczyn A D, De Deyn P P, Clarke C E, Lang A E. A five-year study of the incidence of dyskinesia in patients with early Parkinson's disease who were treated with ropinirole or levodopa. N Engl J Med. 2000; 342:1484-91.), or L-dopa administration with a catechol-O-methyltransferase inhibitor (COMTI) to extend dopamine elimination half-life (Stocchi F, Rascol O, Kieburtz K, Poewe W, Jankovic J, Tolosa E, Barone P, Lang A E, Olanow C W. Initiating levodopa/carbidopa therapy with and without entacapone in early Parkinson disease: the STRIDE-PD study. Ann Neurol. 2010; 68:18-27.) failed to significantly improve the severe LDRC.

The spatial distribution of dopamine and methotrexate during continuous intracerebral microperfusion has also been studied (Sendelbeck S L and Urquhart J. Spatial Distribution of Dopamine, Methotrexate and Antipyrine During Continuous Intracerebral Microperfusion. Brain Research 1985; 328:251-258). The infusion was made in the brain tissues, more particularly into the mid thalamic region of diencephalon, with an Alzet 2001 mini-osmotic pump filled with dopamine hydrochloride and sodium methotrexate dissolved in deoxygenated artificial cerebrospinal fluid containing sodium fluorescein. The mini-osmotic pump was filled with the solution at least 16 h prior to implantation. However, under these conditions, oxygen will necessarily penetrate into the pump and render the dopamine toxic. Moreover, the study was only made in order to analyze the diffusion of different drugs according to their lipid solubility and polarity, without any therapeutic intention.

The continuous release of dopamine from a mesoporous matrix of TiO2 has been disclosed in MX 2012012559. Dopamine is embedded into the matrix which is produced by a sol gel method. However, said matrix must be implanted into the caudate nucleus of the brain, said implantation being invasive and not convenient at all for the patient. Moreover, this continuous release of dopamine from the mesoporous matrix only enables the symptoms of Parkinson's disease to be controlled, without producing any neuroprotective effect.

Another therapeutic strategy relates to a continuous dopamine infusion directly into the striatum or the lateral ventricle in animals.

Yebenes et al (1987) evaluated the effect of dopamine or dopamine agonists by intracerebroventricular infusion on rats with unilateral lesions of the nigro striatal pathway and MPTP-treated monkeys. The infusion was made in the cerebral lateral ventricle ipsilateral to the lesion with a catheter connected to an Alzet 2001 pump filled with dopamine in different vehicles such as sodium metabisulfite. Sodium metabisulfite was used in order to reduce dopamine's auto-oxidation. It was observed that motor symptoms decreased and that intracerebral concentrations of dopamine increased. However, contralateral rotation was induced by infusion of dopamine or dopamine agonists with a peak 2 days after the implantation and a slow decrease over a period of 5 days infusion. This effect shows that the continuous infusion induces a tachyphylaxis effect, supported by the reduction in the number of DA-receptors in infused animals. This means that the treatment induces an adaptation phenomenon with a progressive loss of efficiency. It is thus required to progressively increase the dopamine dosage in order to keep a maximal efficiency (de Yebenes J G1, Fahn S, Lovelle S, Jackson-Lewis V, Jorge P, Mena M A, Reiriz J, Bustos J C, Magarinfos C, Martinez A. Continuous intracerebroventricular infusion of dopamine and dopamine agonists through a totally implanted drug delivery system in animal models of Parkinson's disease. Mov Disord. 1987; 2:143-58).

Moreover, a problem of oxidation was observed. Dopamine autoxidation induces formation of quinones and free radicals which are highly cell toxic. This auto-oxidation of dopamine induces oxidation of the surrounding tissues and cell walls. This oxidation has been shown to induce neurotoxicity and consequently could act on the worsening of Parkinson's disease. This problem of auto-oxidation was reduced but remained when dopamine was dissolved in sodium metabisulfite. Moreover, sodium metabisulfite induces tolerance problems such as allergic reaction to sulfites. Besides, a worsening of neuronal degeneration has been shown to be induced by the use of sulfite on pyramidal neurons (Akdogan I, Kocamaz E, Kucukatay V, Yonguc N G, Ozdemir M B, Murk W. Hippocampal neuron number loss in rats exposed to ingested sulfite. Toxicol Ind Health. 2011; 27:771-8.). This suggests a possible toxicity of sodium metabisulfite in Parkinson's disease model.

The treatment studied in Yebenes et al. was only a symptomatic therapy and was not able to achieve a protection of the dopaminergic neurons in the human substantia nigra and in the striatum.

EP3142651B1 discloses a pharmaceutical solution comprising at least dopamine for use in Parkinson's disease which is kept under anaerobic conditions from its formulation to its administration.

EP3453388A1 discloses a pharmaceutical solution consisting of dopamine hydrochloride dissolved in saline solution, wherein the solution has a pH between 5.5 and 7 and wherein the solution is injectable, free of oxygen and free of preservative agent, and wherein the saline solution consists of water and monosodium or disodium phosphate, sodium, potassium, calcium or magnesium chloride or mixtures of such salts.

However, the inventors found that the dopamine solution disclosed in EP3142651B1 and EP3453388A1 may not be stored over a prolonged period of time at a temperature of 37° C. because of stability problems, which is an inconvenient when using the solution with a pump that is in permanent contact with the skin.

Therefore, there is thus still a need in the art for a treatment of medical conditions linked to low levels of dopamine, especially Parkinson's disease or Parkinson's disease syndromes, that does not present the above-mentioned drawbacks.

More particularly, there is a need for a stable pharmaceutical composition capable of being administered with a pump and which allows for a treatment of medical conditions linked to low levels of dopamine, especially Parkinson's disease or Parkinson's disease syndromes.

### SUMMARY OF THE INVENTION

The inventors have now found that the above drawbacks can be overcome when dopamine or a pharmaceutically acceptable salt thereof, preferably dopamine hydrochloride, is dissolved in water for injection and the resulting pharmaceutical injectable solution has a pH between 3.0 and 5.5, and having an oxygen content equal to or lower than 0.008% (8 ppm).

A first aspect of the invention is directed to a pharmaceutical injectable solution comprising dopamine or a pharmaceutically acceptable salt thereof, preferably dopamine hydrochloride, dissolved in water for injection, wherein the solution has a pH between 3.0 and 5.5, and has an oxygen content equal to or lower than 0.008% (8 ppm).

A second aspect of the invention pertains to the pharmaceutical injectable solution of the invention for use in treating medical conditions linked to low levels of dopamine in a subject in need thereof.

### DETAILED DESCRIPTION OF THE INVENTION

### Definitions

In the following, terms as used herein are defined in their meaning.

The term “about” or “ca.” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also referred to as weight-by-weight percent (w/w %).

As used herein, the terms “effective amount” or “therapeutically efficient amount” of a compound refer to an amount of the compound that will elicit the biological or medical response of a subject, for example, ameliorate the symptoms, alleviate conditions, slow or delay disease progression, or prevent a disease.

As used herein, the term “low levels of dopamine” refers to a dopamine level not enough to ensure normal dopaminergic neurotransmission. This is clinically manifested by the appearance of disturbances of automaticity, particularly motor automaticity, and in particular by the presence of akinesia (i.e. delay in the execution of voluntary movement, but especially automatic movement), bradykinesia (i.e. abnormally slow and rare movement, especially in their automatic component, hypertonia and sometimes the occurrence of resting tremor.

### Pharmaceutical Injectable Solution Comprising Dopamine

The present disclosure relates to a pharmaceutical injectable solution comprising dopamine or a pharmaceutically acceptable salt thereof, preferably dopamine hydrochloride, dissolved in water for injection, wherein the solution has a pH between 3.0 and 5.5, and has an oxygen content equal to or lower than 0.008% (8 ppm).

Indeed, the present invention is based on the unexpected findings that, when dopamine is in a pharmaceutical injectable solution dissolved in water for injection at a pH between 3.0 and 5.5 and wherein the oxygen content is equal to or lower than 0.008% (8 ppm), it is stable for being administered with a pump to a subject while allowing treating medical conditions linked to low levels of dopamine.

Thanks to this specific combination of pH and water for injection, the aqueous solution of dopamine is stable for at least 7 days, preferably 14 days, more preferably 21 days, even more preferably 28 days at 37° C. and thus may be stored anaerobically in a pump which is in permanent contact with the skin over such prolonged periods of time. The solution of the present invention is thus suitable for use according to the present invention.

The pharmaceutical solution of the invention is pharmaceutically acceptable, i.e. is generally safe, non-toxic, and neither biologically nor otherwise undesirable and includes that which is acceptable for human pharmaceutical use.

Dopamine is a sympathomimetic amine vasopressor and is the naturally occurring immediate precursor of norepinephrine. Dopamine is a key neurotransmitter in the brain. Dopamine may be in the form of its free base (4-(2-aminoethyl)benzene-1,2-diol) as well as its pharmaceutical acceptable salts, such as e.g. its hydrochloride.

The term “pharmaceutically acceptable salts” refers to any salt obtained from dopamine, said salt having a slightly similar biological activity compared to the biological activity of said compound of the invention. Dopamine is an amine and may thus forms acid addition salts. Suitable acid addition salts are formed from acids which form non-toxic salts. Examples of such acids are hydrochloric acid, hydrobromic acid, sulfuric acid, phosphoric acid, methanesulfonic acid, acetic acid, fumaric acid, succinic acid, lactic acid, citric acid, tartaric acid, and maleic acid, of which hydrochloric acid, hydrobromic acid, sulfuric acid, phosphoric acid, and acetic acid are preferred. Hence, suitable pharmaceutically acceptable salts are dopamine hydrobromide, dopamine sulfate, dopamine phosphate, dopamine methanesulfonate, dopamine acetate, dopamine fumarate, dopamine succinate, dopamine, dopamine lactate, dopamine citrate, dopamine tartrate, and dopamine maleate, of which the hydrochloride, hydrobromide, sulfate, phosphate, and acetate are preferred. More preferably, the pharmaceutically acceptable salt is dopamine hydrochloride. Pharmaceutically acceptable salts may, for example, be obtained using standard procedures well known in the field of pharmaceuticals. In a particular embodiment, the pharmaceutical injectable solution according to the invention comprise a pharmaceutically acceptable salt of dopamine.

According to the invention, dopamine or a pharmaceutically acceptable salt thereof, preferably, dopamine hydrochloride, is dissolved in water for injection (WFI), a vehicle which is pharmaceutically acceptable for a formulation capable of being injected. “water for injection” (WFI) designates water of extra high quality without significant contamination as defined by pharmacopeial groups worldwide, for example Ph. Eur. 10. Monograph 04/2017:0169.

When dopamine or a pharmaceutically acceptable salt thereof, preferably dopamine hydrochloride, is dissolved in water for injection (WFI), dopamine thus obtained gives a stable acidic solution, having a pH comprised between 3.0 and 5.5, preferably between 3.3 and 5.0, more preferably between 3.5 and 4.5, and even more preferably about 4.0. Thus, the pharmaceutical solution of the invention is in the form of an aqueous solution.

The pharmaceutical injectable solution according to the invention has an oxygen content equal to or lower than 0.008% (8 ppm). In an embodiment, the pharmaceutical solution has an oxygen content equal to or lower than 0.0007% (7 ppm), preferably equal to or lower than 0.0006% (6 ppm), more preferably equal to or lower than 0.0005% (5 ppm), even more preferably of about 0.0002% (2 ppm).

Such oxygen content can be obtained by any methods known in the art, for example by deoxygenation with inert gas such as nitrogen, freons, argon, xenon, (36)-krypton or neon or any other gaz. To this end, the dopamine, preferably dopamine hydrochloride dissolved in water for injection can be performed in inert atmosphere as described in FR0114796.

In an embodiment, the concentration of dopamine in the solution is at least 50 mg/mL, preferably between 50 mg/mL and 1000 mg/mL, more preferably about 100, 150, 200, 300, 400, 500, 600, 700, 800, 900 mg/mL, even more preferably between 100 mg/mL and 400 mg/mL.

In a specific embodiment, when dopamine is dopamine hydrochloride, a concentration of 10 mg/mL for example requires the addition of salt to be isotonic and then capable of being administered to a subject. However, when the concentration is above 50 mg/mL, the pharmaceutical solution is naturally hyperosmolar. The addition of any salt, such as sodium chloride, will further increase the osmolarity of said solution and may be deleterious to the pharmaceutical solution. Preferably, there is no addition of sodium chloride in the pharmaceutical solution especially when the pharmaceutical solution comprises a pharmaceutically acceptable salt of dopamine which is dopamine hydrochloride.

Advantageously, the pharmaceutical injectable solution of the invention is free of preservative agent. By “preservative agent” is meant all molecules, peptides, salts or other compounds which have an antioxidant effect or which is essential to preserve dopamine and other compounds constituting the pharmaceutical solution of the invention.

In a preferred embodiment, the pharmaceutical injectable solution is essentially consisting of dopamine hydrochloride dissolved in water for injection, wherein the solution has a pH between 3.0 and 5.5, preferably between 3.3 and 5.0, more preferably between 3.5 and 4.5, and even more preferably about 4.0, and has an oxygen content equal to or lower than 0.008%, preferably equal to or lower than 0.0007% (7 ppm), more preferably equal to or lower than 0.0006% (6 ppm), even more preferably equal to or lower than 0.0005% (5 ppm), and even more preferably of about 0.0002% (2 ppm). In a specific embodiment, the pharmaceutical injectable solution is essentially consisting of dopamine hydrochloride dissolved in water for injection, wherein the solution has a pH about 4.0, and has an oxygen content of about 0.0002% (2 ppm). By “essentially consisting of” it is meant only the components cited and their eventual impurities, i.e. no addition of any other component.

The pharmaceutical solution comprising dopamine is injectable, i.e. is formulated for a parenteral administration. When the pharmaceutical composition is injectable, it is administered by parenteral route, that is, using a needle (usually a hypodermic needle) and a syringe, or by the insertion of an indwelling catheter. Examples of parenteral administration include intravenous, intramuscular, intranasal, transdermal, submucosal, intrathecal, subcutaneous, intraperitoneally, intraocular, intra-cerebral, for example brain intra-ventricular, etc.

Also provided herein is a pharmaceutical injectable solution adapted to be stored in a vial and which is stable for at least 1 month, preferably 2 months, more preferably 3 months, even more preferably 4 months, even more preferably 5 months, even more preferably 6 months, even more preferably 9 months, even more preferably 12 months, even more preferably 18 months, and even more preferably 24 months at 5° C.

Also provided herein is a pharmaceutical injectable solution adapted to be stored in an anaerobical pump and which is stable for at least 7 days, preferably 14 days, more preferably 21 days, even more preferably 28 days at 37° C.

### Methods of Treatment Using Said Pharmaceutical Injectable Solution

In a second aspect of the invention, the pharmaceutical injectable solution of the present disclosure is for use in treating medical conditions linked to low levels of dopamine in a subject in need thereof.

In other words, the invention also pertains to a method of treating medical conditions linked to low levels of dopamine wherein a therapeutically efficient amount of the pharmaceutical injectable solution of the present disclosure is administered to a subject in need thereof.

The invention also relates to the use of the pharmaceutical injectable solution of the present disclosure for the manufacture of a medicament for treating medical conditions linked to low levels of dopamine in subject in need thereof.

The form (especially the concentration) of the pharmaceutical injectable solution, the route of administration, the dosage and the regimen naturally depend upon the severity of the illness, the age, weight, and sex of the subject.

The term “treatment”, “treating” and derived terms mean reversing, alleviating, stopping or preventing medical conditions linked to low levels of dopamine. The term “treatment” also refers to a prophylactic treatment which can delay the onset of medical conditions linked to low levels of dopamine.

The terms “patient”, “subject”, “individual”, and the like, are used interchangeably herein, and refer to a human, more particularly a human over 45 years old, more preferably over 50 years old. In some embodiments, the patient, subject or individual in need of treatment includes those who already have the disease, condition, or disorder, i.e. medical conditions linked to low levels of dopamine.

In a preferred embodiment, the medical conditions linked to low levels of dopamine is selected from the group consisting of Parkinson's disease, Parkinson's disease syndromes, restless legs syndrome, depression, schizophrenia and attention deficit hyperactivity disorder (ADHD), neurodegeneration with brain iron accumulation and other vascular or degenerative brain diseases with doparesponsive parkinsonism, and genetic disorders affecting the enzymes of synthesis or metabolism. More preferably, the medical conditions linked to low levels of dopamine is Parkinson's disease or Parkinson's disease syndromes.

In an embodiment, less than or equal to 3 mL, preferably between 1 mL and 3 mL, preferably about 2 mL, of said pharmaceutical injectable solution is administered daily to the subject.

In another embodiment, at least 25 mg, preferably between 25 mg and 500 mg, more preferably between 50 mg and 400 mg, even more preferably between 75 mg and 300 mg, even more preferably between 100 mg and 250 mg, even more preferably about 200 mg of dopamine is administered daily to the subject.

Advantageously, said pharmaceutical injectable solution is suitable for brain intraventricular administration. More specifically, said pharmaceutical solution is adapted to be administered into the right lateral ventricle, preferably at the entrance of the interventricular foramen so that the pharmaceutical solution can be administered into the third ventricle.

Indeed, the present inventors have surprisingly discovered that an administration at the entrance of the interventricular foramen is possible, in particular by placing the catheter into right lateral ventricle at the entrance of the interventricular foramen, which enables the pharmaceutical injectable solution to be directly administered into the third ventricle. Thus, it allows the bilateral concentration of dopamine into the striatum through the ventricle walls and the subventricular area (SVZ). This administration considerably reduces motor complications, whereas dopamine is laterally concentrated into frontal region and caudate nucleus when administered into the frontal region of the brain, which would be less advantageous with respect to motor complications and development of psychoses.

To this end and in order to perform the administration under anaerobic conditions, the pharmaceutical injectable solution according to the invention is adapted to be administered with an anaerobical pump.

By “anaerobical pump” is meant any device which enables a controlled release of the solution of the invention and which do not degrade the anaerobia of said solution by exposing it to oxygen. Typically, said pump must be compatible with the present invention, and is in particular able to anaerobically deliver a dopamine solution to the desired site of administration. For example, a SYNCHROMED II pump (commercialized by Medtronic in Ireland), a iPRECIO pump (commercialized by ALZET in the USA) or an ALZET pump (commercialized by Alzet), or Siromedes pump (commercialized by Tricumed in Germany), or Prometra II pump (commercialized by Flowonix in the USA) can be used for this purpose. The Prometra II pump (commercialized by Flowonix) is suitable for humans and can thus be preferably used on a human patient. This pump allows complete anaerobic conditions and an excellent stability of the dopamine. Hence, the use of these pumps extremely reduces the risk of oxidation or auto-oxidation of dopamine. The benefit/risk balance for the use of dopamine in the treatment of Parkinson's disease was negative before the development of these anaerobical pumps. This pump is in permanent contact with the skin once set up in the subject in need thereof. Indeed, the pump is implanted at the subcutaneous paraumbilical level and above the rectus muscles. The pump is therefore at an intracorporeal temperature of 37° C. It is connected to a subcutaneous catheter up to the frontal level where the catheter penetrates the brain over a few centimeters up to the right ventricular frontal horn.

The administration of the solution of the invention under anaerobic conditions can also be performed by any other method known by the person skilled in the art.

Inventors have found that above a concentration of dopamine in the solution of 100 mg/mL, the pharmaceutical injectable solution is hyperosmolar, i.e. above 295 mOsm/L, which may not be tolerated by the subject's body. Indeed, the osmolarity depends on the volume of the injectable solution to be injected and on the injection compartment in which said injectable solution is injected and its volume. Generally speaking, intolerance problems occur with intravenous injectable solutions when the osmolarity exceed 600 mOsm/L. Inventors have found that injecting to a subject a pharmaceutical injectable solution having a concentration above 100 mg/mL of dopamine is possible only if the rate of injection is controlled in such a way that the formulation injected into the subject's body, preferably into the cerebrospinal fluid (CSF), is directly diluted so that it is acceptable and tolerated by the subject. For example, a volume of 2 mL of 100 mg of dopamine hydrochloride, thus a concentration of 100 mg/mL dopamine hydrochloride in the pharmaceutical injectable solution, will be diluted in the volume of the cerebrospinal fluid, which is known to be about 150 mL, with a production rate of cerebrospinal fluid of around 20 mL/h, and an elimination rate of the cerebrospinal fluid which increase with the volume of the cerebrospinal fluid, so that the osmolarity of the cerebrospinal fluid after the administration of the pharmaceutical injectable solution is close to 295 mOsm/L, i.e. between 295 mOsm/L and 350 mOsm/L, which is acceptable and do not present a risk for the subject.

Hence in an embodiment, the pharmaceutical injectable solution comprising dopamine hydrochloride has an osmolarity of between 800 mOsm/L and 6400 mOsm/L, preferably 1200 mOsm/L and 4800 mOsm/L, more preferably between 1600 mOsm/L and 3200 mOsm/L, more preferably about 1600 mOsm/L.

In certain aspect, the pharmaceutical injectable solution is administered to the subject at a rate between 1 mL/day and 3 mL/day, preferably about 2 mL/day.

In another embodiment, the pharmaceutical injectable solution for use is administered to the subject with an anaerobical pump at a flow rate between 0.04 mL/h and 0.125 mL/h, preferably between 0.06 mL/h and 0.10 mL/h, more preferably about 0.08 mL/h.

Thus, it is unexpected to administer a pharmaceutical injectable solution which is far beyond the maximum recommended osmolarity value, i.e. 600 mOsm/L, and it is thanks to selection of the specific concentration of the pharmaceutical injectable solution and of the flow rate of administration, especially with the anaerobical pump, that the administration became possible and acceptable according to the recommendations.

The doses used for the administration can be adapted as a function of various parameters, and in particular as a function of the mode of administration used, of the relevant pathology, or alternatively of the desired duration of treatment.

The present invention also provides a pharmaceutical injectable solution and its use as described above, wherein said pharmaceutical solution is continuously administered with dose variations. Preferably, said pharmaceutical solution is administered with a predominant diurnal dose or with an exclusive diurnal dose.

“Predominant diurnal dose” means that the nocturnal dose is lower than the diurnal dose, preferably at least 70% lower than the diurnal dose, more preferably at least 80% lower than the diurnal dose, more preferably at least 90% lower than the diurnal dose.

By “an exclusive diurnal dose” is meant that there is no nocturnal dose.

Said administration protocol can be easily carried out by using an anaerobical pump as described above, for example a Prometra II pump (commercialized by Flowonix).

In specific embodiment, the pharmaceutical injectable solution as described above is administered with the following dosage regimen:
a continuous diurnal dose,
optionally, a bolus administered on morning, and
optionally, at least a bolus when required, and/or
a continuous nocturnal dose lower than the diurnal dose, preferably the nocturnal dose is between 1% and 50% of the diurnal dose, even more preferably between 2% and 30% of the diurnal dose, even more preferably between 2.5% and 10% of the diurnal dose.

By “bolus” is meant a single, relatively large dose of the pharmaceutical solution of the invention that is administered in order to achieve an immediate effect. Preferably, the bolus is in the same way as above described. A bolus is administered on morning and optionally when required, i.e. when the patient is in need of an immediate effect of the treatment.

The inventors have discovered that this administration protocol allows the determination of a minimal efficient dose which can vary from one patient to another. Motor and non-motor symptoms of Parkinson's disease are treated without any of the side effects (dyskinesias, fluctuations, psychosis . . . ), which usually occur with peripheral administration of dopaminergic treatments (i.e oral pulsatile administration of L-dopa, subcutaneous administration of apomorphine, jejunal administration of a L-dopa gel) and autoxidation's risks observed with central (intracerebroventricular) administration of aerobic dopamine. These complications or side effects can be stopped or even prevented if the treatment with anaerobic dopamine according to the invention is administrated before the occurrence of such complications. Typically, the use of an anaerobical pump allows determining a minimal efficient dose which is adapted to each case.

By a “minimal efficient dose” is meant a sufficient amount to be effective, at a reasonable benefit/risk ratio applicable to any medical treatment. It will be understood, however, that the total daily usage will be decided by the attending physician within the scope of sound medical judgment. The specific minimal efficient dose for any particular patient in need thereof will depend upon a variety of factors including the age, body weight, general health, sex and diet of the patient, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved. The doses can also vary according to the dopasensitivity of the patient. For example, it has previously been observed a ratio from 1/100 to 1/300 between the required dose administrated per os and the dose administrated with an intracerebroventricular (ICV) route (e.g. morphine, baclofene). Further until now, it was well established in the literature following preclinical trials in monkeys that a dose of 40 mg/day of dopamine was sufficient to be effective in humans (Caroline Moreau, et al., Intraventricular dopamine infusion alleviates motor symptoms in a primate model of Parkinson's disease, Neurobiology of Disease, Volume 139, 2020, 104846; M. K. Home et al., Intraventricular Infusion of Dopamine in Parkinson's Disease, Ann Neurol 1989; 26: 792-794). However, the inventors discovered that the estimated dose was totally underdosed and that it was necessary to double or even triple this dose, i.e. at least 100 mg/day, in order to obtain a sufficient efficacy i.e. lowering appearance of disturbances of automaticity, particularly motor automaticity, preferably the presence of akinesia, dyskinesia, bradykinesia, hypertonia and resting tremor, and lowering appearance of behavior and cognition disturbances, such as apathy, anxiety, sleep disorders.

### FIGURES

FIG. 1: Study design of the clinical trial. D means day; the timelines may be slightly modified depending on the patient's health status (e.g. number of days in hospital) and dosing satisfaction (e.g. length of titration period).

FIG. 2: Impact of A-dopamine on motor fluctuations and dyskinesia measured using the patients' diaries.

### EXAMPLES

Hereinafter, the present disclosure is described in more details and specifically with reference to the examples, which however are not intended to limit the present invention.

### Example 1: Optimal pH Determination for Compatibility with the Delivery Pump

The inventors found that the dopamine solution comprising dopamine hydrochloride at pH 5.5 dissolved in saline solution (0.9% NaCl) is not stable enough to allow sufficient storage in the pump at 37° C. The inventors thus experienced lowering the pH of the solution in order to be as acidic as possible.

The objective of the trial is to follow the aging of the pumps in operation over a period of one year by filling them with saline solutions (0.9% NaCl) buffered at pH 4, pH 3 and pH 2. Checks on the proper functioning of the pumps include:
Monitoring the daily delivery rate of the filling solution set at the start of the test,
The research of a possible degradation of the various materials constituting the pumps by the dosage of metallic elements in the solutions of fillings.

### Materials and Methods

Saline solutions (0.9% NaCl) were buffered using a 1:10 dilution of 2 mol/I (2N) HCL solution to obtain test solutions at pH 4, pH 3 and pH 2. The commercial saline solutions (0.9% NaCl) used for the preparation of buffered solutions had the following references:
B Braun Medical, ref. 432 858, lot No 20362404, exp. August 2023,
Versol, ref. 600019, lot No C0669, exp. April 2023,
Versol, ref. 600019, lot No C0826A01, exp. November 2023.

The three pumps were placed in an oven at 37° C. Each pump was filled with a buffered saline solution (NaCl 0.9%): one at pH 4, one at pH 3 and one at pH 2. The density of the three buffered solutions was checked to make sure that there was no difference in filling volume between the three pumps.

The pumps were set at a flow rate of 0.9 ml per day with a nominal filling of 20 ml. A new filling was done on average every 21 days after a complete emptying of the tank. A part of the recovered solutions was used for the research and the determination of the released metallic elements. The solutions analyzed were those recovered after 7 days, 15 days, 29 days, 91 days, 238 days, 315 days and 352 days.

### Determination of the Density of Buffered Saline Solutions

The density of each buffered solution at pH 4, pH 3 and pH 2 was determined by weighing using the cut-flask method at a temperature of 37° C.±2° C. The cut-vial method comprised the following steps:

On a balance with a resolution of 0.0001 g, three weighings were performed:
25 ml pycnometer filled with buffered NaCl solution to the gauge line: M_{NaCl}
25 ml pycnometer filled with water up to the gauge line: M_{w}
Empty and dry 25 ml pycnometer: M_{E}

Before each weighing, the balance was tared. The density of the buffered solution is given by the following formula:

$$d = \frac{MWFI - ME}{M W - ME}$$

The result of the densities is expressed in kg/m3 in the following table:

TABLE 1

| Reference of | | Content | Average value |
|---|---|---|---|
| buffered solutions | Test | kg/m³ | kg/m³ |
| pH 2 | No1 | 998 | 998 |
| | No2 | 998 | |
| | No3 | 998 | |
| pH 3 | No1 | 1000 | 1000 |
| | No2 | 1000 | |
| | No3 | 1000 | |
| pH 4 | No1 | 1002 | 1002 |
| | No2 | 1002 | |
| | No3 | 1002 | |

The density values obtained for the three buffered solutions are very close to each other. The average value of the 3 solutions is equal to 1000±2 kg/m3.

### Monitoring the Operation of the Pumps

Each time the pumps were filled, various checks were carried out:

Checking the remaining volume in the pump: Sampling and measuring the volume remaining in the pump.

Checking the pump operation: Reading of the indication on the INQUIRY control module.

### Visual Inspection:

External appearance of the equipment pump and catheter,
The appearance of the solution.

Over one year of operation, no visual degradation of the three pumps was noted and all the solutions recovered are clear and limpid.

### Research and Determination of Metallic Elements in the Recovered Solutions

A panoramic analysis was performed by inductively coupled plasma mass spectrometry (ICP-MS, Perkin Elmer, NEXION 300X). After a calibration of the mass scale by a representative standard solution, the solutions recovered after 7 and 15 days of operation were measured, leading to estimated contents for all elements of the periodic table accessible by this technique. This analysis is not quantitative, but it easily allows to identify the elements significantly present and which will be the subject of subsequent analyses.

Depending on the elements detected and the concentration levels observed by the exploratory panoramic analysis, two analytical techniques are implemented:
the element silicon is quantitatively determined on a one-tenth dilution of the solutions by plasma emission spectrometry (ICP-OES, HORIBA, ACTIVA-M) against a calibration line established extemporaneously.
The other detected elements are quantitatively determined by inductively coupled plasma mass spectrometry (ICP-MS) on the solutions after mineralization in the presence of a mixture of appropriate acids in a microwave oven. The elements selected were: barium, chromium, copper, iron, molybdenum, nickel, lead, titanium, tungsten and zinc.

The three filling solutions buffered to pH 4, pH 3 and pH 2 were stored in brown glass vials in the oven at 37° C. next to the three pumps for the duration of the study. These are referred to as “test blanks”. For each analysis of the solutions recovered during the operation of the pumps, an analysis was performed in parallel on these “test blank” solutions. The results in metallic elements obtained for these “test blanks” were deduced from the results obtained on the test solutions.

The determination of metallic elements was performed on the solutions recovered after 7 days, 15 days, 29 days, 91 days, 238 days, 315 days and 352 days of operation.

TABLE 2
Barium contents obtained as a function
of operating time and expressed in μg/kg

| Number of days | Solution | Solution | Solution |
|---|---|---|---|
| of operation | at pH 4 | at pH 3 | at pH 2 |
| 7 | 928 | 750 | 1032 |
| 15 | 342 | 356 | 557 |
| 29 | 52 | <50 | 83 |
| 91 | <20 | <20 | 44 |
| 238 | <20 | <20 | <20 |
| 315 | <20 | <20 | <20 |
| 352 | 21 | <20 | <20 |

TABLE 3
Chromium contents obtained as a function
of operating time and expressed in μg/kg

| Number of days | Solution | Solution | Solution |
|---|---|---|---|
| of operation | at pH 4 | at pH 3 | at pH 2 |
| 7 | <50 | <50 | 1579 |
| 15 | <50 | <50 | 155 |
| 29 | <20 | <20 | <20 |
| 91 | <20 | <20 | <20 |
| 238 | <20 | <20 | <20 |
| 315 | <20 | <20 | <20 |
| 352 | <20 | <20 | <20 |

TABLE 4
Copper contents obtained as a function
of operating time and expressed in μg/kg

| Number of days | Solution | Solution | Solution |
|---|---|---|---|
| of operation | at pH 4 | at pH 3 | at pH 2 |
| 7 | <10 | 23 | 111 |
| 15 | <10 | 42 | 47 |
| 29 | <10 | 37 | 78 |
| 91 | <20 | <20 | 206 |
| 238 | <20 | <20 | 127 |
| 315 | <20 | <20 | 188 |
| 352 | <20 | <20 | 122 |

TABLE 5
Iron contents obtained as a function of
operating time and expressed in μg/kg

| Number of days | Solution | Solution | Solution |
|---|---|---|---|
| of operation | at pH 4 | at pH 3 | at pH 2 |
| 7 | <50 | <50 | 5649 |
| 15 | <50 | 227 | 728 |
| 29 | <50 | 244 | 197 |
| 91 | <100 | <100 | 1527 |
| 238 | 245 | <100 | 109 |
| 315 | <100 | <100 | <100 |
| 352 | <100 | <100 | <100 |

TABLE 6
Molybdenum contents obtained as a function
of operating time and expressed in μg/kg

| Number of days | Solution | Solution | Solution |
|---|---|---|---|
| of operation | at pH 4 | at pH 3 | at pH 2 |
| 7 | <10 | <10 | 146 |
| 15 | <10 | <10 | 24 |
| 29 | <10 | <10 | <10 |
| 91 | <10 | <10 | 43 |
| 238 | <10 | <10 | <10 |
| 315 | <10 | <10 | <10 |
| 352 | <10 | <10 | <10 |

TABLE 7
Nickel contents obtained as a function
of operating time and expressed in μg/kg

| Number of days | Solution at | Solution at | Solution at |
|---|---|---|---|
| of operation | pH 4 | pH 3 | pH 2 |
| 7 | 404 | 27235 | 57200 |
| 15 | 562 | 15280 | 23226 |
| 29 | 272 | 16650 | 7365 |
| 91 | 89 | 59 | 751 |
| 238 | <10 | 75 | 695 |
| 315 | 12 | 43 | 301 |
| 352 | 73 | <10 | 279 |

TABLE 8
Lead contents obtained as a function of
operating time and expressed in μg/kg

| Number of days | Solution at | Solution at | Solution at |
|---|---|---|---|
| of operation | pH 4 | pH 3 | pH 2 |
| 7 | <10 | 15 | 101 |
| 15 | <10 | 69 | 42 |
| 29 | <10 | 22 | 39 |
| 91 | <10 | <10 | 190 |
| 238 | <10 | 65 | 200 |
| 315 | <10 | 100 | 188 |
| 352 | <10 | 123 | 378 |

TABLE 9
Titanium contents obtained as a function
of operating time and expressed in μg/kg

| Number of days | Solution at | Solution at | Solution at |
|---|---|---|---|
| of operation | pH 4 | pH 3 | pH 2 |
| 7 | <50 | <50 | 749 |
| 15 | <50 | <50 | 1056 |
| 29 | <50 | <50 | 587 |
| 91 | <20 | <20 | 363 |
| 238 | <20 | <20 | 133 |
| 315 | 30 | <20 | 69 |
| 352 | <20 | <20 | 76 |

TABLE 10
Silicon contents obtained as a function
of operating time and expressed in μg/kg

| Number of days | Solution at | Solution at | Solution at |
|---|---|---|---|
| of operation | pH 4 | pH 3 | pH 2 |
| 7 | 22580 | 8662 | 9772 |
| 15 | 19835 | 14969 | 10599 |
| 29 | 10305 | 4066 | 11829 |
| 91 | 4850 | 3032 | 5217 |
| 238 | Not | 3177 | 5605 |
| | measurable | | |
| 315 | 2838 | 2048 | 4169 |
| 352 | 4144 | 3281 | 5243 |

TABLE 11
Tungsten contents obtained as a function
of operating time and expressed in μg/kg

| Number of days | Solution at | Solution at | Solution at |
|---|---|---|---|
| of operation | pH 4 | pH 3 | pH 2 |
| 7 | 971 | 248 | 136 |
| 15 | 2138 | 320 | 275 |
| 29 | 8741 | 412 | 262 |
| 91 | 1557 | 1698 | 394 |
| 238 | 1536 | 330 | 300 |
| 315 | 319 | 158 | 188 |
| 352 | 205 | 159 | 164 |

TABLE 12
Zinc contents obtained as a function of
operating time and expressed in μg/kg

| Number of days | Solution at | Solution at | Solution at |
|---|---|---|---|
| of operation | pH 4 | pH 3 | pH 2 |
| 7 | Not | Not | Not |
| | measured | measured | measured |
| 15 | Not | Not | Not |
| | measured | measured | measured |
| 29 | Not | Not | Not |
| | measured | measured | measured |
| 91 | <200 | <200 | 1362 |
| 238 | <200 | 1079 | 1337 |
| 315 | <200 | 1235 | 1625 |
| 352 | 574 | 1600 | 2101 |

Zinc was not determined on the first three samples because it was not detected as migrant element in the panoramic analyses performed by inductively coupled plasma mass spectrometry (ICP-MS).

### Results

For the Pump Filled with the pH 4 Solution

A strong migration of the elements barium, nickel, tungsten and silicon during the first month of operation was observed, then a very clear decrease to a constant or even non-detectable level for some elements. The other elements were not significantly detected.

For the Pump Filled with the pH 3 Solution:

A strong migration of the elements barium, iron, nickel, silicon during the first month of operation was observed, then a very clear decrease until a constant or even non-detectable content for some elements. The migration of the element lead increased progressively. The migration of the element tungsten was progressive during the first three months and then decreases until a constant content is reached. The migration of the element zinc started after three months of operation and then increased strongly. The other elements were not significantly detected.

For the Pump Filled with the pH 2 Solution:

Migration of the elements barium, chromium, iron, molybdenum, nickel and titanium was stronger than for the pH 4 and pH 3 solution during the first month of operation, then a clear decrease until the content was constant or even non-detectable for certain elements. A strong migration of the element silicon during the first month of operation was observed, then a clear decrease until constant content. The migration of the element tungsten was progressive during the first three months and then decreased until constant content. The copper element migrated constantly. The migration of the element lead increased progressively. The migration of the element zinc started after three months of operation and then increased more strongly than for the solutions at pH 4 and pH 3.

### Conclusion

Lowering the pH of the solution to pH 2, results in massive amounts of metals released from the pump which are above the norms. At pH 3 the pump released transient levels of metals, some of which were above the norms. At pH 4 the levels were acceptable.

### Example 2: 6-Month Stability Study of a Solution of Dopamine Hydrochloride Solution at 100 mg/mL Diluted in Water for Injection at pH 4 and Stored at 5° C.

This stability study was performed on a solution of dopamine hydrochloride packaged anaerobically in 20 mL type I amber glass vials, closed with elastomer stoppers and then crimped. Dopamine hydrochloride is sensitive to light and to oxygen naturally present in the air. The manufacture of the solution and the filling was carried out under anaerobic conditions to avoid the degradation of the active ingredient and to avoid the formation of degradation products and, in particular, of its neurotoxic degradation product, 6-hydroxydopamine (6-OHDA).

The concentration of dopamine is always expressed in average % of the initial concentration (% C0)±standard deviations, in tabular and graphical form.

The pH, absorbance at 320 nm and osmolality of the solutions are expressed with the mean value of the analysis result ±standard deviations, in the form of tables.

Protocol for the Preparation of Dopamine Hydrochloride Solution at 100 mg/mL Diluted in WFI, pH 4 and Filling of 20 mL Glass Vials

The manufacturing steps of the solution were performed in an anaerobic zone under a nitrogenous chamber ( ) with an oxygen level lower than 0.01%. The oxygen level in the chamber is checked before the start of each operation.

Two liters of solution are required to fill 90 vials of 20 mL and are packaged in two 1L volumetric flasks. 100 g of dopamine hydrochloride of European Pharmacopoeia quality (Lot no 62317-250, ref: 005765, INRESA, Bartenheim, France) are dissolved in 1 L of WFI (ref: DE0304, Baxter, Guyancourt, France). After visual control, the two 1L solutions are placed in a beaker to homogenize the concentration. The pH of the solution was already 4.00, so no pH adjustment was performed. The pH is measured using a handheld pH meter (HI9125 with HI1333B semi-micro glass electrode, Hanna instruments, Lingolsheim, France) which is calibrated before use.

The composition of the injection solution (per unit and per batch) is summarized in Table 13.

TABLE 13

| | | Theorical | | Therocial quantity | |
|---|---|---|---|---|---|
| | | quantity by unit | | for a 2 L batch | |
| Components | Quality Standard | (20 mL vial) | | (90 vials of 20 mL) | |
| Dopamine chlorhydrate | Ph. Eur. 0664 | 2000 | mg | 200,00 | g |
| Water for injection | Medecine | q.s. 20 | mL | q.s. 2000 | mL |
| | authorized 34009 | | | | |
| | 358 551 5 2 | | | | |

Vials were also filled under anaerobic conditions. The vials were filled with 20 mL polypropylene Luer-Lock syringes (ref: 300629, Becton Dickinson, Le Pont de Claix, France) fitted with a 0.22 μm porosity filter with a Supor® membrane (ref: HP4642, Pall, Saint-Germain-En-Laye, France) so that the solutions could be filtered and sterilized prior to packaging. The sterilizing filtration was performed with a different filter for each vial in order to take into account the filter manufacturer's guarantee. A beaker of 2 L of solution was required to fill 90 vials (1 batch=90 vials of 20 mL). The vials were then capped in the chamber and crimped, after being removed from the laminar flow hood. The crimping was manually checked and the vials are subject to visual inspection before and after quarantine (15 days to perform the sterility test).

No final sterilization was performed due to the presence of inert gas in the vials, notably nitrogen. The vials were finally labelled and stored at 5° C. for 6 months to perform stability studies.

### Stability Tests

Analyses were performed every 7 days from T0 to T1 month and then every 15 days from T1 month to T6 month. At each analysis time, the concentration pH, osmolality and absorbance at 320 nm were checked on 3 bottles.

### Concentration Measurement by HPLC-UV

The initial concentration of dopamine hydrochloride diluted in water for injection was 101.98±0.28 mg/mL for the pH 4 solution. The percentages of dopamine hydrochloride remaining in the solution relative to the initial concentration C0 at the different study times are displayed Table 14.

TABLE 14

| | |
|---|---|
| Time | Concentration/initial concentration |

| (month) | Average | Standard deviation |
|---|---|---|
| 0 | 100% | 0% |
| 0.25 | 98.15% | 1.24% |
| 0.5 | 100.41% | 0.95% |
| 0.75 | 98.77% | 1.89% |
| 1 | 99.41% | 1.56% |
| 1.5 | 99.34% | 1.32% |
| 2 | 99.74% | 1.44% |
| 2.5 | 100.32% | 0.94% |
| 3 | 100.38% | 1.18% |
| 3.5 | 100.46% | 1.75% |
| 4 | 100.66% | 0.27% |
| 4.5 | 101.57% | 0.45% |
| 5 | 100.01% | 2.24% |
| 5.5 | 98.89% | 2.04% |
| 6 | 100.94% | 1.52% |

During 6 months of storage at 5° C., the concentration of dopamine hydrochloride remained well above our 90% limit. In addition, no traces of 6-OHDA appeared, meaning that this degradation product remains at concentrations below 0.019 μg/mL.

### Measurement of pH

According to the European Pharmacopoeia monograph 2.2.3, the pH was measured by potentiometry, using the pH meter HI9125 with its semi-micro glass electrode HI1333B (Hanna instruments, Lingolsheim, France). It was calibrated before each use with standard solutions. For each dosing time, the pH is measured on 3 vials.

Specifications: The pH should not vary by more or less than 0.5 pH units from the pH of the starting solution.

Table 15 shows the evolution of pH during 6 months of storage at 5° C.

TABLE 15

| | |
|---|---|
| Time | pH measurements |

| (month) | Average | Standard deviation |
|---|---|---|
| 0 | 4.02 | 0.00 |
| 0.25 | 4.05 | 0.02 |
| 0.5 | 4.13 | 0.02 |
| 0.75 | 4.11 | 0.02 |
| 1 | 4.10 | 0.03 |
| 1.5 | 4.11 | 0.03 |
| 2 | 4.12 | 0.02 |
| 2.5 | 4.13 | 0.01 |
| 3 | 4.13 | 0.02 |
| 3.5 | 4.14 | 0.01 |
| 4 | 4.17 | 0.01 |
| 4.5 | 4.20 | 0.01 |
| 5 | 4.27 | 0.03 |
| 5.5 | 4.31 | 0.02 |
| 6 | 4.32 | 0.04 |

During 6 months of conservation, the pH did not change in the glass botties.

### Measurement of Osmolality

According to the European Pharmacopoeia monograph 2.2.35, osmolality was measured using the Fiske Micro osmometer model 210 (Advanced Instruments, Horsham, UK).

For each dosing time, osmolality was measured on 3 vials. Specifications: Osmolality should not vary by more or less than 10 mOsmol/kg from the osmolality of the starting solution.

Table 16 shows the evolution of osmolality during 6 months of storage at 5° C.

TABLE 16

| | |
|---|---|
| Time | Osmolality (mOsmol/kg) |

| (month) | Average | Standard deviation |
|---|---|---|
| 0 | 874 | 2 |
| 0.25 | 875 | 2 |
| 0.5 | 877 | 2 |
| 0.75 | 877 | 1 |
| 1 | 877 | 2 |
| 1.5 | 877 | 2 |
| 2 | 876 | 2 |
| 2.5 | 876 | 1 |
| 3 | 877 | 2 |
| 3.5 | 877 | 2 |
| 4 | 877 | 2 |
| 4.5 | 877 | 1 |
| 5 | 878 | 2 |
| 5.5 | 878 | 1 |
| 6 | 878 | 1 |

This solution is hypertonic to blood plasma and, during 6 months of storage, the osmolality has not changed in the glass vials.

### Solution Staining

The extent of decomposition of dopamine was determined by measuring the absorbance of the respective solution at 320 nm using a UV spectrophotometer (UV2550, Shimadzu, Noisiel, France). For each assay time, the absorbance was measured on 3 vials. The lower the absorbance, the higher is the stability of dopamine.

Table 17 represents the evolution of the absorbance at 320 nm of dopamine hydrochloride solutions at 100 mg/mL at pH4 diluted in WFI during 6 months of storage.

TABLE 17

| | |
|---|---|
| Time | Absorbance at 320 nm |

| (month) | Average | Standard deviation |
|---|---|---|
| 0 | 0.002 | 0.001 |
| 0.25 | 0.003 | 0.001 |
| 0.5 | 0.003 | 0.001 |
| 0.75 | 0.003 | 0.001 |
| 1 | 0.003 | 0.001 |
| 1.5 | 0.004 | 0.002 |
| 2 | 0.006 | 0.001 |
| 2.5 | 0.006 | 0.001 |
| 3 | 0.010 | 0.001 |
| 3.5 | 0.011 | 0.001 |
| 4 | 0.013 | 0.002 |
| 4.5 | 0.014 | 0.006 |
| 5 | 0.020 | 0.003 |
| 5.5 | 0.021 | 0.004 |
| 6 | 0.025 | 0.001 |

During 6 months of storage at 5° C., the solutions remained stable.

### Conclusion

The dopamine hydrochloride solution at 100 mg/mL, pH 4, diluted in water for injection remained stable during 6 months of storage at 5° C. whatever the parameters studied: concentration, pH, osmolality, coloration. There is no detection of 6-OHDA.

### Example 3: Stability Study of a Solution of Dopamine Hydrochloride Solution at 100 mg/mL Diluted in Water for Injection at pH 4 in a Prometra II Pump

For this treatment to be implemented in patient, a stability study of a 100 mg/mL solution of dopamine hydrochloride diluted in water for injection (WFI) adjusted to pH 4 was conducted over 28 days. The pumps were filled using an “anaerobic kit” itself prepared in an anaerobic chamber. Dopamine hydrochloride is sensitive to light, oxygen and temperature. As it degrades, it becomes colored and forms one of the neurotoxic dopamine degradation products, 6-hydroxydopamine (6-OHDA). Instability is recognized as soon as 6-OHDA is detected in the solution, and/or staining occurs.

### Protocol for the Preparation of the Prometra II Pump

The manufacturing steps of the pump packaging were performed in an anaerobic chamber with an oxygen level below 0.01% and a temperature of 37° C.

The pump was immersed in a tub containing lard (pork fat) to mimic the layers of skin and fat that will be on top and underneath the pump when it was implanted in the patient.

The pump was filled with a solution of dopamine hydrochloride 100 mg/mL at pH 4 contained in a 20 mL glass vial (see report “Protocol for the preparation of amber glass vials containing a solution of dopamine hydrochloride 100 mg/mL diluted in water for injection” of example 2). Protocols of the tests carried out for the follow-up of the stability study, and specifications of the tests carried out on the manufactured batches for the manufacture and stability of the vials, were carried out in the open air. After filling, the pump tray was returned to the chamber and the chamber is set to 37° C. and 0.01% oxygen for the study.

### Collection of Samples

The purpose of this manipulation was to program the pump to deliver 0.5 mL of solution per 24 hours. The experiment was performed in anaerobic conditions (0.01% oxygen) and at 37° C. in the chamber. The liquid was collected at the catheter exit in a closed Eppendorf TUBESO® (DNA LoBind Tube 1.5 MI, ref: 0030 108.051, Eppendorf AG, Hamburg, Germany) (containing just a hole on the lid to let the catheter pass through) during the first 24 hours and then analyzed in HPLC-UV and UV spectrophotometry. In addition, the liquid was also collected inside the pump and analyzed by HPLC-UV and UV spectrophotometry. Collection and analysis were performed every 7 days for 4 weeks. A control Eppendorf® tube containing 0.5 mL of 100 mg/mL dopamine hydrochloride solution adjusted to pH 4 was placed next to the recovery Eppendorf® tube. The volume of the control solution was measured at the end of the 24-hour collection period to estimate sample evaporation.

### Results

### Concentration Measurement by HPLC-UV

During the stability study, the concentration of dopamine hydrochloride was monitored by a High-Performance Liquid Chromatography (HPLC) assay coupled with a UV-visible detector. The concentration range of dopamine hydrochloride was validated for a target concentration of 200 μg/mL. This method validation had been performed with a stock solution of dopamine hydrochloride which was diluted in 0.9% NaCl, but our solution of dopamine hydrochloride at 100 mg/mL is diluted in water for injection. To perform the assay, this solution was diluted 1:500 under the same conditions as in the validated method, so the change of diluent did not affect the results of HPLC-UV assays.

Detection at 280 nm for dopamine, 291 nm for 6-OH dopamine (6-OHDA).

N=3 determinations on the collected sample.

Specifications: A proven impact of the studied factors is considered to exist when the concentration of active ingredient is less than 90% of the initial concentration. In addition, the study will be stopped if 6-OHDA is detected.

The initial concentration of diluted dopamine in water for injection is 98.39±1.95 mg/mL. The percentages of dopamine hydrochloride measured in the solution relative to the initial concentration C0 at the different study times inside the pump and at the catheter outlet are displayed in Table 19.

TABLE 19

| | |
|---|---|
| Catheter outlet | Inner pump |

| Time | | Standard | | Standard |
|---|---|---|---|---|
| (days) | Average | deviation | Average | deviation |
| 0 | 100% | 0% | 100% | 0% |
| 7 | 98.80% | 0.23% | 100.20% | 0.79% |
| 14 | 97.01% | 1.60% | 100.37% | 1.17% |
| 21 | 99.99% | 0.91% | 98.45% | 1.30% |
| 28 | 97.63% | 1.89% | 98.84% | 0.93% |

During 28 days of storage at 37° C., the concentration of dopamine hydrochloride remained above 90% of the initial concentration at the catheter outlet and in the Prometra II pump. Moreover, no trace of 6-OHDA appeared during HPLC/UV assays during the 28-day study.

### Limit of Solution Staining

The extent of decomposition of dopamine was determined by measuring the absorbance of the respective solution at 320 nm using a UV spectrophotometer (UV2550, Shimadzu, Noisiel, France). For each determination time, the absorbance was measured on the sample taken from the sample. The lower the absorbance, the higher is the stability of dopamine.

The concentration of dopamine is always expressed as average % of the initial concentration (% C0)±standard deviations, in tabular and graphical form.

The absorbance at 320 nm of the solution is expressed with the value of the analysis result, in tabular form.

Table 20 represents the absorbances at 320 nm of the 100 mg/mL dopamine hydrochloride solution at pH 4.

TABLE 20

| Time | | |
|---|---|---|
| (days) | Catheter outlet | Inner pump |
| 0 | 0.003 | 0.003 |
| 7 | 0.177 | 0.034 |
| 14 | 0.188 | 0.043 |
| 21 | 0.197 | 0.041 |
| 28 | 0.205 | 0.056 |

During 28 days of storage at 37° C., the absorbance at 320 nm remained stable at the catheter outlet and in the Prometra II pump.

### Conclusion

Dopamine hydrochloride solution at 100 mg/mL diluted in water for injection at pH 4 is stable for 28 days in the Prometra II pump (Flowonix) at 37° C. under the conditions tested and can therefore be implemented in patients.

### Example 4: Clinical Trial—Brain Infusion of Dopamine in Parkinson's Disease

Motor and Non-Motor Handicap Between the Oral Phase and A-Dopamine Phase with Moderate Doses

### Patients and Ethical Standards

Two patients were enrolled prospectively in the PD Centre of Excellence, Lille, France (Table 21).

Patients and 2 received moderate doses, i.e. dose less than or equal to 150 mg/day, specifically 99 mg/day (5.5 mg/h), of A-dopamine formulated as a solution of dopamine hydrochloride (50 and 100 mg/mL) dissolved in water for injection at pH 4 in a sterile anaerobic nitrogen isolator (oxygen <0.1%) to avoid degradation by dopamine auto-oxidation, without any other excipient and manufactured by the Central Pharmacy of the University Hospital of Lille.

TABLE 21
Comparison of motor and non-motor handicap between the
oral phase and A-dopamine phase with moderate doses

| | | |
|---|---|---|
| Patient 1 | Patient 2 | Mean ± SD |

Baseline characteristics

| | | | |
|---|---|---|---|
| Age (years) | 53 | 58 | 55.5 ± 2.5 |
| Disease duration | 8 | 11 | 9.5 ± 1.5 |
| (years) | | | |
| Duration of L-dopa | 7 | 10 | 8.5 ± 1.5 |
| (years) | | | |
| Duration of motor | 5/3 | 5/4 | 5 ± 0/3.5 ± 0.5 |
| fluctuations/ | | | |
| dyskinesia (years) | | | |
| LEDD (mg) | 1942.5 | 1010 | 1476 ± 466 |
| Dopamine agonist | Yes | no | |
| IMAO-B (rasagiline) | Yes | yes | |
| ICOMT (entacapone) | | | |

| | | | | | |
|---|---|---|---|---|---|
| | | | | | Change |
| Phase II | Oral | Dopamine | Oral | Dopamine | Mean ± SD |

Motor fluctuations and dyskinesia

| | | | | | |
|---|---|---|---|---|---|
| Dyskinesia (DRS) | 64 | 18 | 62 | 0 | 54 ± 8 |
| MDS-UPDRS | 10 | 6 | 13 | 0 | 8.5 ± 4.5 |
| fluctuations, part IV | | | | | |
| MDS-UPDRS part II | 20 | 12 | 13 | 9 | 6 ± 2 |
| (off periods) | | | | | |
| MDS-UPDRS part II | 7 | 7 | 9 | 7 | 1 ± 1 |
| (on periods) | | | | | |
| MDS-UPDRS part III | 18 | 14 | 12 | 8 | 4 ± 0 |
| (on drug) | | | | | |

One-week actimetry at home

| | | | | | |
|---|---|---|---|---|---|
| Bradykinesia score (50 | 13.2 | 19.45 | 20 | 18.9 | −2.5 ± 3.6 |
| percentile) | | | | | |
| Dyskinesia score (50 | 28.6 | 3.2 | 10.2 | 4 | 15.8 ± 9.6 |
| percentile) | | | | | |
| Fluctuation Dyskinesia | 22 | 13.5 | 12.2 | 7.1 | 12.2 ± 7.1 |
| score | | | | | |

Behavior & cognition

| | | | | | |
|---|---|---|---|---|---|
| MDS-UPDRS part I | 12 | 7 | 13 | 6 | 6.8 ± 1.7 |
| NPI-C | 12 | 11 | 17 | 14 | 5 ± 2 |
| Active impulse control | yes | no | no | no | |
| disorders | | | | | |
| Apathy (LARS) | −23 | −21 | −22 | −22 | 1 ± 1 |
| Parkinson anxiety | 5 | 5 | 8 | 8 | 0 ± 0 |
| scale | | | | | |
| Sleepiness (Epworth) | 5 | 9 | 6 | 6 | 2 ± 2 |
| PD sleep scale | 81 | 91 | 97 | 97 | 5 ± 5 |
| Cognition (MOCA) | 29 | 30 | 26 | 27 | 1 ± 0 |
| Quality of life PDQ-39 | 53 | 30 | 35 | 20 | 19 ± 4 |
| Reduction of LEDD (%) | | 53.2 | | 67 | 60.1% ± 6.9 |
| Dopamine agonist | yes | no | no | no | |
| IMAO-B (rasagiline) | yes | yes | yes | yes | |
| ICOMT (entacapone | | | | | |
| amantadine | | | | | |
| Total duration of A- | | 9 | | 4 | 6.5 ± 2.5 |
| dopamine (months) | | | | | |
| Dopamine in urine | 13082 | 13327 | 9173 | 9023 | 47 ± 197 |

Hoehn & Yahr scale of symptom progression from stages 1-5 (bedridden) (2 indicates a bilateral disorder); Schwab & England activities of daily living from 0-100 (completely independent); MDS-UPDRS: Movement Disorders Society-Unified Parkinson Disease Rating Scale; Off and On drug means with or without L-dopa; L-dopa sensitivity (%) means % improvement on the MDS-UPDRS part Ill after an acute administration of 150% of the morning dose of L-dopa; LEDD: levodopa equivalent daily dose in mg; IMAO-B: inhibitor of monoamine oxidase B; ICOMT: inhibitor of catechol-O-methyl transferase; ICD: previous or current impulse control disorder under dopaminergic agonist. DRS: Dyskinesia Rating Scale total score (dyskinesia+dystonia) (the highest being the worst); Movement Disorders Society—Unified Parkinson Disease Rating Scale (MDS-UPDRS) part I (cognition and behaviour); part II activity of daily living during the good “On” periods and the troublesome “Off” periods; motor handicap (part III): MDS-UPDRS part Ill (On) was assessed at 10 am after the L-dopa dose at 9 am under conditions of both exclusive oral treatment or in association with A-dopamine; fluctuation with dyskinesia and dystonia (part IV) (the highest being the worst); One-week actimetry at home: actimeter (Parkinson Kinetograph™ from GKC) in a wristwatch worn on the most affected side: bradykinesia score (50 percentile: normal value=18.6); Dyskinesia score (50 percentile: normal value=4.3); Fluctuation Dyskinesia Score (estimating the amount of variability in relation to optimal control, i.e. insufficient control (bradykinesia) or overdose (dyskinesia); expected benefit with a reduction in %); NPI-C: Neuropsychiatric Inventory-Clinician version (/798, the highest being the worst); LARS: Lille Apathy Rating scale (−36 to +36, the highest being the worst); Parkinson Anxiety Scale (/48, the highest being the worst); Epworth Sleepiness Scale (/24, the highest being the worst); PD sleep scale (/150, the highest being the best); MOCA: Montreal Cognitive Assessment (/30, the highest being the best). Dopamine in urine in nmol/mmol creatinine.

The inclusion criteria were as follows:
(i) a diagnosis of PD consistent with MDS criteria, at the stage of severe motor and non-motor L-dopa-related complications, including at least 2 h of Off and 1 h of L-dopa-induced dyskinesia not controlled by optimised oral drug therapy (i.e. with at least five doses of L-dopa);
(ii) meeting the criteria for invasive second-line treatment
(iii) subcutaneous continuous apomorphine infusion not sufficiently effective, poorly tolerated, contraindicated or refused;
(iv) patient preferring A-dopamine to the two other existing and validated therapies (i.e. deep brain stimulation (subthalamic or internal pallidum) or levodopa-carbidopa intestinal gel.

The main non-inclusion criteria were:
(i) subject >75-years of age;
(ii) dementia (DSM IV criteria, MDS criteria and MOCA score s22); and
(iii) a decompensated psychiatric disorder using the semi-structured psychiatric M.I.N.I interview.

### Surgery and Implantable System

The catheter (Flowonix™) implantation procedure in the right frontal horn near the interventricular foramen of Monro was performed by the neurosurgical team, with stereotactic placement of the catheter guided by a Renishaw's Neuro Mate robot. Control of the catheter position was done intra-operatively with the O-Arm system. The catheter was attached to the right frontal bone of the skull with the Medtronic stim Lock® system and then tunneled under the skin into the abdominal region, where it was connected to a 20 ml telemetry-adjustable pump delivery system (Prometra II Flowonix), which was buried in a subcutaneous pocket. A postoperative scan was performed within 48 h to verify the absence of haematoma and to confirm correct positioning of the catheter after the end of surgery.

### A-Dopamine Preparation

A-dopamine was formulated as a solution of dopamine hydrochloride (50 and 100 mg/mL) dissolved in water for injection at pH 4 in a sterile anaerobic nitrogen isolator (oxygen <0.1%) to avoid degradation by dopamine auto-oxidation, without any other excipient, and manufactured by the Central Pharmacy of the University Hospital of Lille.

### Preoperative and Postoperative Assessment

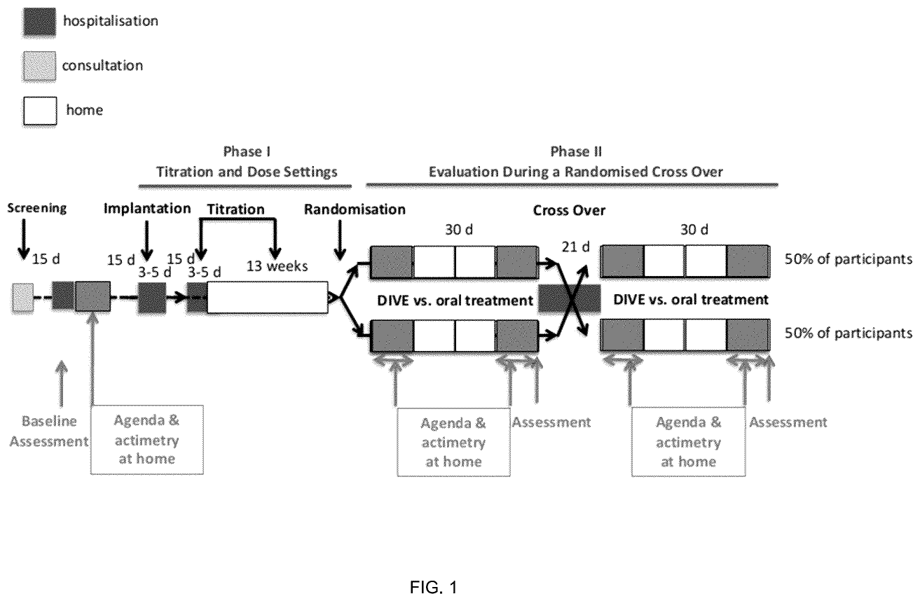

A comprehensive preoperative assessment of L-dopa-induced dyskinesia, L-dopa-related complications, motor, cognitive and behavioural symptoms was performed at the baseline visit. The evaluation was carried out sequentially with a phase I of titration and a phase II using a crossover of 1 month as compared to the usual optimised oral treatment with the same comprehensive assessment before and after each period (FIG. 1).

### Phase I: Titration and Dose Settings

An initial hospital titration was performed for 3-5 days with an increase from 1 to 18 mg/day (up to 1 mg/h over 18 h daytime), and titration was then continued in the outpatient setting under real-life conditions, with a weekly increase of 18 mg (1 mg/h over 18 h), until the dose was reached which achieved satisfactory motor control. The pump was refilled every 7-15 days as needed to ensure and control the quality of A-dopamine.

For the patients' comfort, oral treatment was decreased slowly during titration to avoid a transient motor aggravation. The patient was assessed weekly in consultation for changes in pump rate and monitoring of neurological, psychiatric and cardiovascular safety (pulse, blood pressure, electrocardiogram). The ideal moderate dose was defined as the dose that will result in a marked attenuation of L-dopa-related complications with a substantial reduction in oral therapy. The maximum tolerated dose without adverse reactions was also determined. When the patient was titrated and satisfied, he moved on to phase 2. The pump was refilled every 7-15 days as required to ensure and control the quality of A-dopamine.

### Phase I/: Evaluation During the Randomised Crossover Study

A randomised, controlled, open-label study was performed in a crossover design of two 4-week periods separated by a therapeutic switch of 21 days (see FIG. 1). The two patients were randomised into the two following treatment sequences, either (i) period 1: A-dopamine treatment with residual oral treatment; and (ii) period 2: exclusive optimised oral treatment per os; or the reverse sequence. A comprehensive assessment was performed including 1-week evaluations at home with diaries completed by the patient and 1-week actimetry at home with a wristwatch (Parkinson Kinetograph™, GKC) 15 worn on the most affected side.

The previous treatment was stopped at the end of period 1 and replaced by the new treatment, without discontinuity and in a progressive manner, so that the patient was always treated. After stabilisation and in the absence of adverse reactions, the patient returned home and started the period 2 assessments 1 week later to eliminate potential residual effects of the first treatment. The objectives were to evaluate, using the same preoperative comprehensive assessment, the impact of continuous intracerebroventricular administration of A-dopamine on L-dopa-related complications, motor, cognitive and behavioural symptoms compared to optimised oral medical therapy, except for the acute challenge with L-dopa. One-week evaluations at home were carried out with diaries completed by the patients in the first and fourth week of each period. The diary offers eight possibilities to be checked by the patient each hour of the day between sleep, dyskinesia (severe, moderate, mild), Off period (severe, moderate, mild) and perfect control. A learning process was carried out with the patient and his neurologist before and during the titration of phase 1. One-week actimetry at home was performed simultaneously with a wristwatch (Parkinson Kinetograph™, GKC) worn on the most affected side, in the first and fourth week of each period, in order to record the bradykinesia score (50 percentile: normative value=18.6); dyskinesia score (50 percentile: normative value=4.3) and the fluctuation in dyskinesia score (estimating the amount of variability in relation to optimal control, i.e. insufficient control (bradykinesia) or overdose (dyskinesia)).

An independent data safety monitoring board reviewed all of the data weekly. The total amount of dopamine was measured in a 24 h urine collection in conditions of exclusive oral treatment or in association with A-dopamine. After randomisation, patient 2 was started on the 1-month exclusive oral treatment phase followed by the A-dopamine phase, and the reverse for patient 1.

### Results

### Slow Titration and Moderate Dose Settings in Phase I

General anaesthesia and stereotactic implantation did not result in any adverse reactions.

Titration was started slowly for the first patient from 1 mg/day up to 10 mg/day and then up to 50 mg/day with concentrations of 10 mg/ml, and then from 54 mg/day up to 90 mg/day with a concentration of 50 mg/ml. For patient 2, titration started immediately with 1 mg/h over the 18 h of treatment (5 am to 11 pm) (i.e. increase of 18 mg each week). No adverse reaction related to A-dopamine was noted. The colour of A-dopamine withdrawn from the pumps always remained below the predefined threshold for oxidation. The first benefit reported by all patients was the great reduction in L-dopa-induced dyskinesia and severe off period, even before the first reduction of L-dopa

### Rapid Titration in Phase I

Rapid titration over 1 day with hourly dose changes was performed for the first patient. The hourly increase of 1 mg/h to 10 mg/h resulted in no adverse reaction. The average dose of 10 mg/h avoided the need of oral L-dopa from 8:30 am to 5 pm. Doses from 12 mg/h to 18 mg/h resulted in dose-dependent drowsiness and nausea and orthostatic hypotension from 15 mg/h in patient 1. Importantly, no hallucinations, hypomania or dyskinesia were noted even at a very high dose of A-dopamine.

Phase II with Moderate Doses During 2 Months

Patients received a daytime dose of A-dopamine of 99 mg (i.e. 5.5 mg/h from 5 am to 11 pm). The night-time dose was slightly different among the patients depending on the need to control nocturnal Off periods, from 3 and 2 mg respectively for patients 1 and 2. The most striking benefit was the great reduction in L-dopa-induced dyskinesia and motor fluctuations as observed from actimetry, the diaries and the scales (see Table 1). The nocturnal dopamine requirement was very low and very effective over the periods of under-dosing. The impact of A-dopamine on behaviour and cognition showed either improvement or no aggravation. No patient had the induction or worsening of impulse control disorder or hypomania, and no worsening of anxiety, depression, apathy, sleepiness, sleep quality and cognition. Conversely, both patients reported less impulse control disorders and less irritability with a positive impact on quality of life and a reduction of about 60% of oral treatment. There appeared to be no difference in dopamine in urine under A-dopamine when compared to the period of exclusive oral L-dopa treatment in all patients. Both patients wished to remain on A-dopamine long-term. It was then decided to resume titration at a higher dose, to further assess the treatment potential.

### Evaluation of Higher Dose on Long Term

Four patients received exclusive oral treatment and A-dopamine at moderate and high doses.

Moderate daytime dose was 5.5 mg/h (i.e. 99 mg daytime) A-dopamine formulated as a solution of dopamine hydrochloride (2 and 10 mg/mL) dissolved in water for injection at pH 4 in a sterile anaerobic nitrogen isolator (oxygen <0.1%) to avoid degradation by dopamine auto-oxidation, without any other excipient and manufactured by the Central Pharmacy of the University Hospital of Lille and high daytime dose was 11.5 mg/h (i.e. 207 mg/24 h) A-dopamine formulated as a solution of dopamine hydrochloride (50 and 100 mg/mL) dissolved in water for injection at pH 4 in a sterile anaerobic nitrogen isolator (oxygen <0.1%) to avoid degradation by dopamine auto-oxidation, without any other excipient and manufactured by the Central Pharmacy of the University Hospital of Lille. The nocturnal A-dopamine doses remained unchanged between moderate and high daytime doses (patient 1: 20 mg; patients 2 and 3: 10 mg; patient 4: 2.5 mg).

During two-weeks, patients completed home diaries comparing oral treatment and A-dopamine at moderate and high doses. The diary of motor fluctuations and dyskinesia offers eight possibilities to be checked by the patient each hour of the day between sleep, dyskinesia (severe, moderate, slight), Off period (severe, moderate, slight) and perfect control.

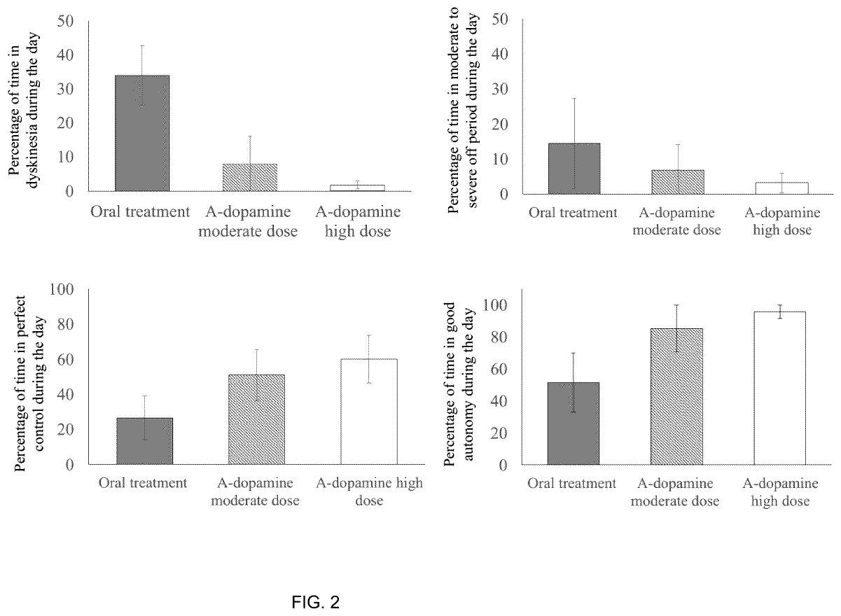

The percentage of daytime in severe, moderate or slight dyskinesia (upper left), in severe, moderate Off periods (upper right), in perfect control (lower left) and in perfect control or slight Off (autonomy, lower right) are presented with histograms of the means with standard deviation values and the value for each patient (FIG. 2). The follow-up time is 21 months for patient 1, 16 months for patient 2, 8 months for patient 3 and 6 months for patient 4.

A greater benefit with a dose-effect was noted with the increase in dose up to 10.5-11.5 mg/h over the daytime period (5 a.m. or 7 a.m.-11 p.m.) on the quasi disappearance of dyskinesia and periods of moderate to severe Off, and a reduction of 70% of oral treatment (FIG. 2). The follow-up time is 8 months for patient 1 and 6 months for patient 2.

Regarding the safety profile, no adverse reaction was observed under A-dopamine with doses <11.5 mg/h and titration increases <2 mg/h. However, daytime doses >11.5 mg/h and rapid titration >2 mg/h resulted in excessive drowsiness in patients 1, 2 and 4, and mild orthostatic hypotension in patients 1 and 3. No hallucinations, recurrence of addictive behaviour, hypomania or dyskinesia were noted even with highest doses, but rather an inhibitory action on motor skills and behaviour. Increasing the nocturnal dose from 2 to 5 mg over the effective dose resulted in nocturnal awakenings that disappeared with decreasing dose, highlighting the low nocturnal dopamine requirement.

### Discussion

Since the identification of nigro-striatal dopamine depletion in PD, it seems rational to compensate for it by continuous administration, following the example of opotherapy with insulin in diabetes. The control of oxidation of this neurotransmitter by its manufacture and delivery under strict anaerobic conditions helps to avoid tachyphylaxis with a stable and great improvement in dopa-responsive clinical symptoms over several months. The use of a high-tech pump allows fine tuning of the dose and the respect of circadian rhythm to further improve the personalised benefit and ergonomics. One of the most striking observations was the important safety of intracerebroventricular infusion of A-dopamine. Indeed, the highest chronic doses, above the efficient doses, only caused drowsiness and nausea without any dyskinesia, hallucinations, hypomania or other behavioural disorders.

There was a dose effect of A-dopamine on efficacy on L-dopa-related complications together with the oral L-dopa reduction. The highest dose allowed the almost complete disappearance of L-dopa-induced dyskinesia and periods of severe Off despite a remaining dose of 50 mg of L-dopa every 2 to 3 hours. Remarkably, the two patients remained very stable for most of the day, between perfect control and slight Off. The same effect was observed on non-motor symptoms, with a good safety profile under A-dopamine (less irritability, no hypomania and no impulse control disorder) when compared with L-dopa and dopaminergic agonists. This benefit was maintained on long term from 6 to 8 months.

During the first years of PD progression, oral L-dopa treatment improves motor symptoms by about 30-50% without L-dopa-induced dyskinesia. Then after a mean of 5 years, L-dopa induces a pathological state of L-dopa-related complications with L-dopa-induced dyskinesia during which the patients wrongly seemed to have a full recovery of PD symptoms at the expense of L-dopa-induced dyskinesia. However, chorea with hypotonia of L-dopa-induced dyskinesia masks the residual bradykinesia and rigidity. A-dopamine administered by the intracerebroventricular route does not induce the same clinical effect as L-dopa administered orally, without the induction of a pathological state with dyskinesia.

It remains to be better defined how A-dopamine acts on dopaminergic neurotransmission. A-dopamine crosses the ependymal layer and follows the flow through the glymphatic system to reach the central nervous system in general and the striatum, in particular. A-dopamine can then continuously stimulate post-synaptic receptors. A-dopamine is also recaptured by pre-synaptic dopaminergic neurons of the SNpc through the dopamine transporter. However, the off target is also probable with notably recaptured by norepinephrine transporters. Urine dopamine levels did not fall during A-dopamine administration despite a large reduction in oral L-dopa. This suggests that a small proportion of A-dopamine infused into the cerebrospinal fluid is flushed into the venous system and then eliminated via the kidneys.

These results support the clinical feasibility of brain infusion of A-dopamine as a solution of dopamine hydrochloride dissolved in water for injection at pH 4 in a sterile anaerobic nitrogen isolator (oxygen <0.1%) without any other excipient, in patients with PD at the stage of L-dopa-related complications.

### Example 5: Clinical Trial—Brain Infusion of Dopamine in Parkinson's Disease with High Dose

Motor and Non-Motor Handicap Between the Oral Phase and A-Dopamine Phase with Higher Dose

### Patient and Ethical Standards

One patient was enrolled prospectively in the PD Centre of Excellence, Lille, France (Table 22).

The Patient received a high dose of 280 mg/day, i.e. a dose of more than or equal to 150 mg/day, specifically 240 mg during daytime and 40 mg during nighttime, of A-dopamine formulated as a solution of dopamine hydrochloride (100 mg/mL) dissolved in water for injection at pH 4 in a sterile anaerobic nitrogen isolator (oxygen <0.1%) to avoid degradation by dopamine auto-oxidation, without any other excipient and manufactured by the Central Pharmacy of the University Hospital of Lille.

### Table 22: Patient Profile

TABLE 22
Patient profile

| | Patient |
|---|---|
| Age (years) | 54 |
| Disease duration (years) | 9 |
| Duration of L-dopa (years) | 9 |
| Duration of motor fluctuations/dyskinesia | 4/4 |
| (years) | |
| LEDD (mg) (Levodopa Equivalent daily dose = | 2412 |
| the dose of all the dopaminergic treatments | |
| before treatment by A-Dopamine) | |

During 2 weeks, the patient completed home diaries comparing oral treatment (Levodopa) and A-dopamine treatment at high dose, i.e. 2 weeks of home diary with oral treatment and 2 weeks of home diary with A-dopamine treatment. The diary of motor fluctuations and dyskinesia offers eight possibilities to be checked by the patient each hour of the day between sleep, dyskinesia (severe, moderate, slight), Off period (severe, moderate, slight) and perfect control. Table 23 summarizes the result in the following categories: time in good autonomy, time in perfect control, time in dyskinesia, time in severe dyskinesia, and time in severe off, wherein time in perfect control is a subset of time in good autonomy.

TABLE 23
Comparison of motor and non-motor handicap between
the oral phase and A-dopamine phase with high doses

| Phase II | Oral | A-Dopamine |
|---|---|---|
| Home diary | | |
| Time in good autonomy: On with slight | 68% | 73% |
| dyskinesia or slight off | | |
| Time in perfect control: On without | 46% | 54% |
| dyskinesia | | |
| Time in dyskinesia | 11% | 3% |
| Time in severe dyskinesia | 1% | 0% |
| Time in severe off | 20% | 12% |
| Reduction of LEDD (%) | | 60% |

The A-dopamine treatment allows to reduce by 60% the oral treatment by Levodopa.

Regarding the safety profile, no adverse reaction was observed under A-dopamine with higher dose of 280 mg/day. Only transient adverse event of nausea occurred with A-dopamine during titration i.e. escalation of the dose until 280 mg/day (2 months and a half of titration). After titration no adverse event was observed. No adverse event of neurosurgery, No ECG or blood medication, No adverse event with the device (pump for administration), No severe adverse event with A-Dopamine.

Two further patients with a similar patient profile received the same high-dose A-Dopamine treatment and they completed home diaries in the same way for both oral treatment with Levodopa and high-dose A-Dopamine treatment. The two further patients showed exactly the same response profile as the first patient reported above.

### Conclusion

There was a dose effect of A-dopamine on efficacy on L-dopa-related complications together with the oral L-dopa reduction. The 280 mg/day dose allowed the almost complete disappearance of L-dopa-induced dyskinesia and good reduction of periods of severe Off. Remarkably, the patient remained very stable for most of the day, between perfect control and slight Off. Further, at this dose, after titration, no adverse event has been noted (nausea, dyskinesia, hallucinations, hypomania or other behavioural disorders) which is important to be noted. These results support the clinical feasibility of brain infusion of A-dopamine as a solution of dopamine hydrochloride dissolved in water for injection at pH 4 in a sterile anaerobic nitrogen isolator (oxygen <0.1%) without any other excipient, in patients with PD at the stage of L-dopa-related complications.

### Example 6: Comparative Example of Example 3 of US2005070613 A1

The Applicant repeated example 3 (paragraph [0091]-[0102]) of application US2005070613 A1, where dopamine was prepared in water for injection at a concentration of 10 mg/mL. According to the European Pharmacopoeia monograph 2.2.35 (11^{th} edition (11.3) of July 2023), osmolality was measured using the Fiske Micro osmometer model 210 (Advanced Instruments, Horsham, UK). The osmolality measured is of 97 mOsmol/kg, thus hypoosmolar i.e. below the osmolality of an isotonic solution. As hypoosmolar solutions are not suitable for administration to humans, the 10 mg/mL solution of example 3 of US2005070613 A1 is not suitable for administration either. Hence, example 3 teaches away from using water for injection for preparing injectable dopamine solutions.

1. A pharmaceutical injectable solution comprising dopamine or a pharmaceutically acceptable salt thereof, dissolved in water for injection, wherein the solution has a pH between 3.0 and 5.5, and has an oxygen content equal to or lower than 0.008% (8 ppm).

2. The pharmaceutical injectable solution according to claim 1, wherein the solution has a pH of about 4.0.

3. The pharmaceutical injectable solution according to claim 1, wherein the solution contains dopamine or a pharmaceutically acceptable salt thereof in a concentration of at least 50 mg/mL.

4. The pharmaceutical injectable solution according to claim 1, in which there is no addition of sodium chloride, especially when the pharmaceutically acceptable solution comprises a pharmaceutically acceptable salt which is dopamine hydrochloride.

5. The pharmaceutical injectable solution according to claim 1, wherein the pharmaceutical injectable solution is free of preservative agent.

6. A method of treatment of medical conditions linked to levels of dopamine, comprising a step of administering a therapeutically effective amount of the pharmaceutical injectable solution according to claim 1 to a subject in need thereof.

7. The method of treatment of according to claim 6, wherein the medical condition linked to low levels of dopamine is selected from the group consisting of Parkinson's disease, Parkinson's disease syndromes, restless legs syndrome, depression, schizophrenia and attention deficit hyperactivity disorder (ADHD), neurodegeneration with brain iron accumulation and other vascular or degenerative brain diseases with doparesponsive parkinsonism, and genetic disorders affecting the enzymes of synthesis or metabolism.

8. The method of treatment according to claim 6, wherein less than or equal to 3 mL of said pharmaceutical injectable solution is administered daily to the subject.

9. The method of treatment according to claim 6, wherein at least 25 mg of dopamine is administered daily to the subject.

10. The method of treatment according to claim 6, wherein the pharmaceutical injectable solution is administered into a brain ventricle.

11. The method of treatment according to claim 6, wherein the pharmaceutical injectable solution is administered with an anaerobical pump.

12. The method of treatment according to claim 11, wherein the pharmaceutical injectable solution is administered to the subject with an anaerobical pump at a flow rate between 0.04 mL/h and 0.125 mL/h.

13. The method of treatment according to claim 6, wherein said solution is continuously administered to the subject with dose variations.

14. The method of treatment according to claim 6, wherein said solution is administered with a predominant diurnal dose or with an exclusive diurnal dose.

15. The method of treatment according to claim 6, wherein said solution is administered with the following dosage regimen:
a continuous diurnal dose,
optionally, a bolus administered on morning, and
optionally, at least a bolus when required, and/or
a continuous nocturnal dose lower than the diurnal dose.

16. The method of treatment according to claim 6, wherein the pharmaceutical injectable solution is administered into the right lateral ventricle.

17. The method of treatment according to claim 6, wherein the pharmaceutical injectable solution is administered at the entrance of the interventricular foramen.
